(19) United States
(12) Patent Application Publication
(10) Pub. No.: US 2024/0285203 A1
(43) Pub. Date: Aug. 29, 2024

(54) DETERMINATION APPARATUS

(71) Applicant: NEC Corporation, Minato-ku, Tokyo (JP)

(72) Inventors: Yuji Ohno, Tokyo (JP); Toshinori Hosoi, Tokyo (JP); Masahiro Kubo, Tokyo (JP)

(73) Assignee: NEC Corporation, Minato-ku, Tokyo (JP)

(21) Appl. No.: 18/026,005

(22) PCT Filed: Sep. 17, 2020
(86) PCT No.: PCT/JP2020/035293

§ 371 Date: Mar. 13, 2023

(51) Int. Cl. A61B 5/16; A61B 5/00; A61B 5/11; A61G 7/05

(52) U.S. Cl. CPC A61B 5/165; A61B 5/1123; A61B 5/7275; A61B 5/7278; A61G 7/05; A61B 2562/0219; A61G 2203/20

(57) ABSTRACT

An agitation determination apparatus includes an acquisition unit 521 configured to acquire body motion data according to a motion of a body of a target and vital data according to a state of the body of the target, a calculation unit 522 configured to calculate a body motion score serving as an index for determining an agitation state based on the body motion data acquired by the acquisition unit 521 and calculate a vital score serving as an index for determining the agitation state based on the vital data acquired by the acquisition unit 521, and a determination unit 523 configured to determine the agitation state of the target based on the body motion score and the vital score calculated by the calculation unit 522.

### TECHNICAL FIELD

The present invention relates to a determination apparatus, a determination method, and a recording medium.

### BACKGROUND ART

When a patient is in an agitated state, a risk of extubation, needle dislodgement, decannulation, tumble, a fall, and the like increases, and the patient may be, for example, injured as a result thereof. In this regard, there have been known techniques for determining a predictive sign of agitation to reduce such a risk and a possibility.

Examples of known documents discussing the techniques for determining a predictive sign of agitation include Patent Literature 1. Patent Literature 1 discusses a biological information processing system including a determination unit and an estimation unit. According to Patent Literature 1, the determination unit determines discrimination information indicating whether or not a condition of a patient has changed in comparison with a normal state based on features of biological information of the patient. Then, the estimation unit estimates countermeasure information for the patient based on the discrimination information determined by the determination unit and countermeasure prediction parameters learned in advance. Further, Patent Literature 1 discloses a heartbeat and the like as one example of the biological information, and discloses an agitation score indicating a possibility that the patient is in an agitated state as one example of the discrimination information.

Further, examples of related documents include Patent Literature 2. Patent Literature 2 discusses a monitoring system that monitors a patient based on image data of the patient. For example, Patent Literature 2 discusses a monitoring system including an image analysis unit for detecting motion events of the patient from the image data, an evaluation unit for classifying the detected motion events into delirium-typical motion events and non-delirium-typical motion events, and a delirium determination unit for determining a delirium score indicating a likelihood and/or strength of delirium of the patient from the duration, intensity, type, location and/or occurrence of the delirium-typical motion events.

### CITATION LIST

Patent Literature 1: International Publication No. 2019/073927
Patent Literature 2: Japanese Unexamined Patent Application Publication (Translation of PCT Application) No. 2014-528314

### SUMMARY OF INVENTION

### Technical Problem

In the case of the technique discussed in Patent Literature 1, it is difficult to correctly recognize the agitation state of the patient, as seen from the fact that, for example, the patient may be agitated although the agitation score determined based on the biological information of the patient has a low value. Further, in the case of the technique discussed in Patent Literature 2, the analysis is made based on the image data acquired from a camera. Therefore, it is difficult to recognize the patient's predictive sign of agitation.

Under these circumstances, one of objects of the present invention is to provide a determination apparatus, a determination method, and a recording medium capable of solving the problem that it is difficult to appropriately determine the agitation state of the patient.

### Solution to Problem

To achieve the above-described object, according to one aspect of the present disclosure, a determination apparatus is configured to include
an acquisition unit configured to acquire body motion data according to a motion of a body of a target and vital data according to a state of the body of the target,
a calculation unit configured to calculate a body motion score serving as an index for determining an agitation state based on the body motion data acquired by the acquisition unit and calculate a vital score serving as an index for determining the agitation state based on the vital data acquired by the acquisition unit, and
a determination unit configured to determine the agitation state of the target based on the body motion score and the vital score calculated by the calculation unit.

Further, according to another aspect of the present disclosure, a determination method is configured to cause a computer to
acquire body motion data according to a motion of a body of a target and vital data according to a state of the body of the target,
calculate a body motion score serving as an index for determining an agitation state based on the acquired body motion data,
calculate a vital score serving as an index for determining the agitation state based on the acquired vital data, and
determine the agitation state of the target based on the calculated body motion score and vital score.

Further, according to another aspect of the present disclosure, a recording medium records a program for causing a computer to realize processing including
acquiring body motion data according to a motion of a body of a target and vital data according to a state of the body of the target,
calculating a body motion score serving as an index for determining an agitation state based on the acquired body motion data,
calculating a vital score serving as an index for determining the agitation state based on the acquired vital data, and
determining the agitation state of the target based on the calculated body motion score and vital score.

### Advantageous Effects of Invention

By being configured in the above-described manner, the present invention allows the agitation state to be appropriately determined.

### BRIEF DESCRIPTION OF DRAWINGS

FIG. 1 is a diagram illustrating an example of the overall configuration of an agitation determination system according to a first exemplary embodiment of the present disclosure.

FIG. 2 is a block diagram illustrating an example of the configuration of a sensor apparatus illustrated in FIG. 1.

FIG. 3 is a block diagram illustrating an example of the configuration of a bed terminal illustrated in FIG. 1.

FIG. 4 is a block diagram illustrating an example of the configuration of an agitation determination apparatus illustrated in FIG. 1.

FIG. 5 is a diagram illustrating one example of information stored in a storage unit illustrated in FIG. 4.

FIG. 6 is a diagram illustrating one example of body motion data included in sensing data illustrated in FIG. 4.

FIG. 7 is a diagram illustrating one example of vital data included in the sensing data illustrated in FIG. 4.

FIG. 8 is a diagram illustrating one example of a body motion score illustrated in FIG. 4.

FIG. 9 is a diagram illustrating one example of a vital score illustrated in FIG. 4.

FIG. 10 is a diagram for illustrating an example of processing by an agitation state determination unit.

FIG. 11 is a diagram for illustrating the example of the processing by the agitation state determination unit.

FIG. 12 is a flowchart illustrating examples of operations of the agitation determination apparatus.

FIG. 13 is a block diagram illustrating another example of the configuration of the agitation determination apparatus.

FIG. 14 is a diagram illustrating one example of information stored in a storage unit illustrated in FIG. 13.

FIG. 15 is a diagram illustrating the hardware configuration of a determination apparatus according to a second exemplary embodiment of the present disclosure.

FIG. 16 is a block diagram illustrating an example of the configuration of the determination apparatus.

### DESCRIPTION OF EMBODIMENTS

### First Exemplary Embodiment

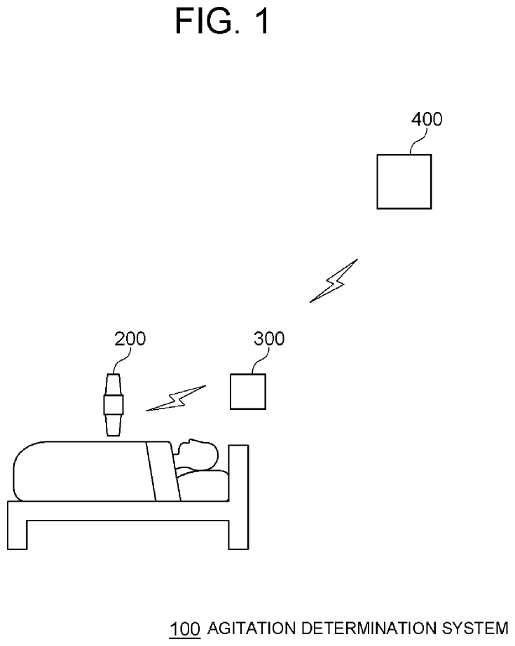

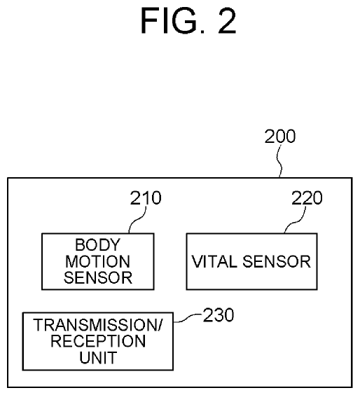

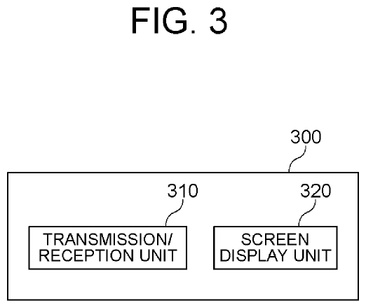

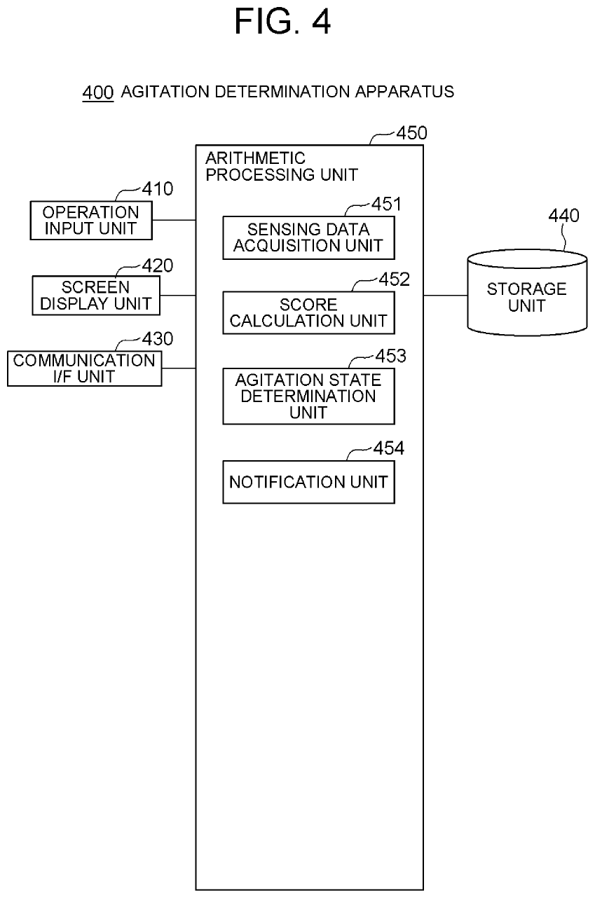

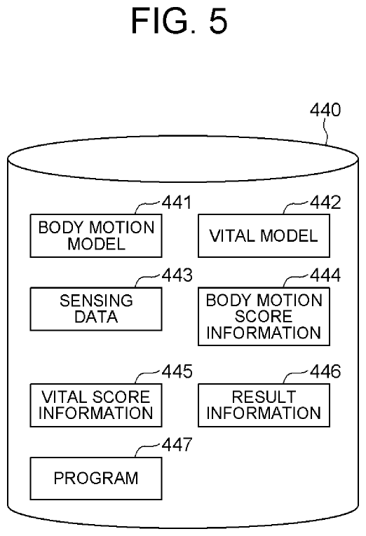

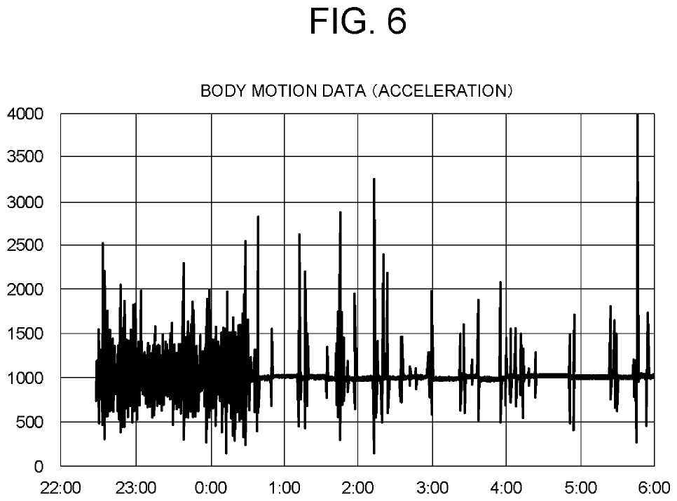

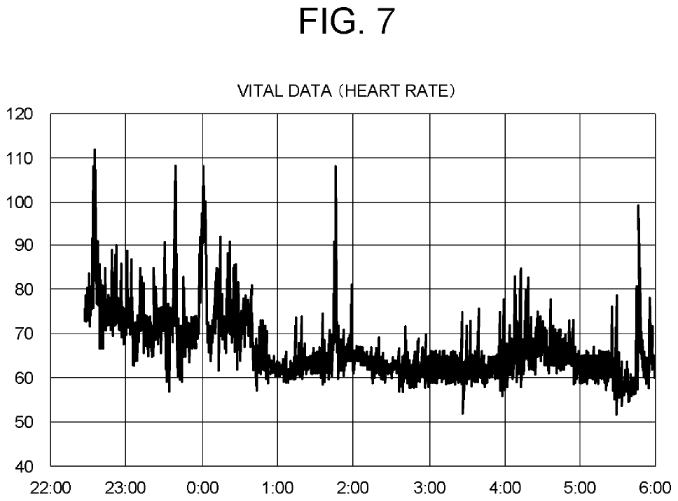

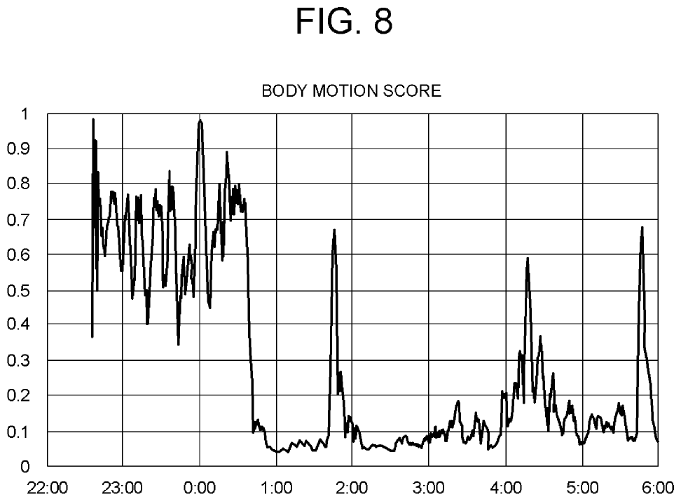

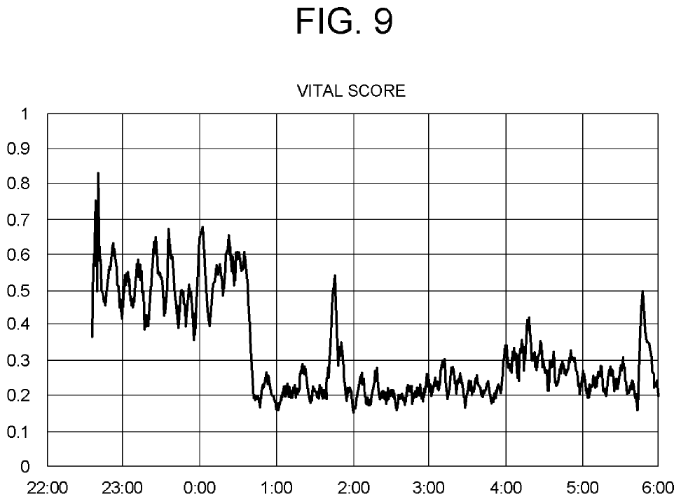

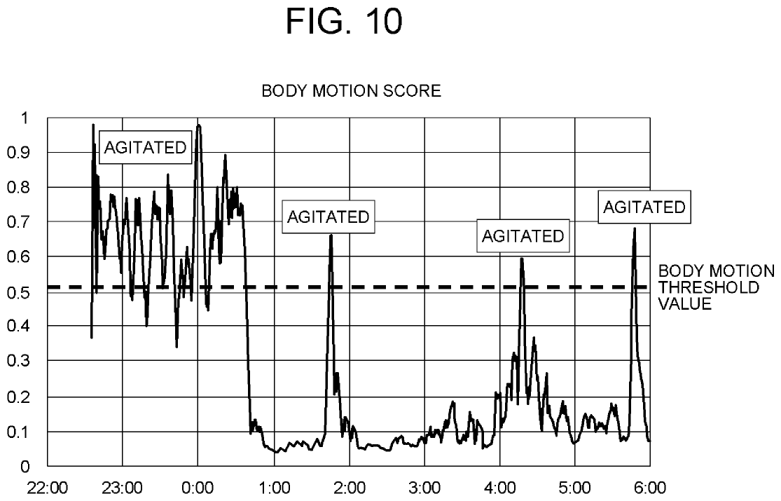

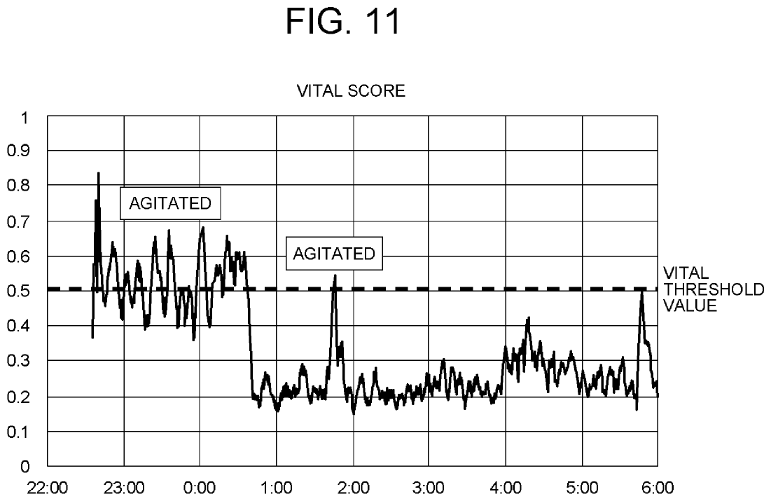

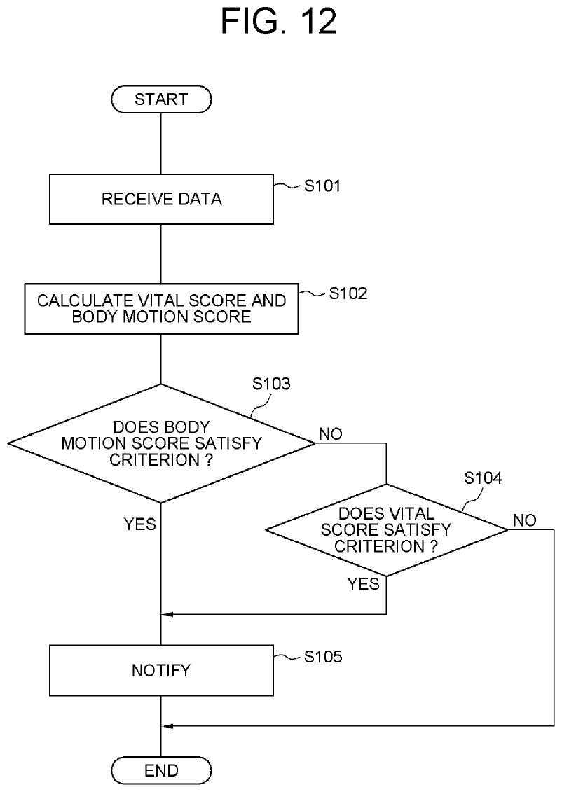

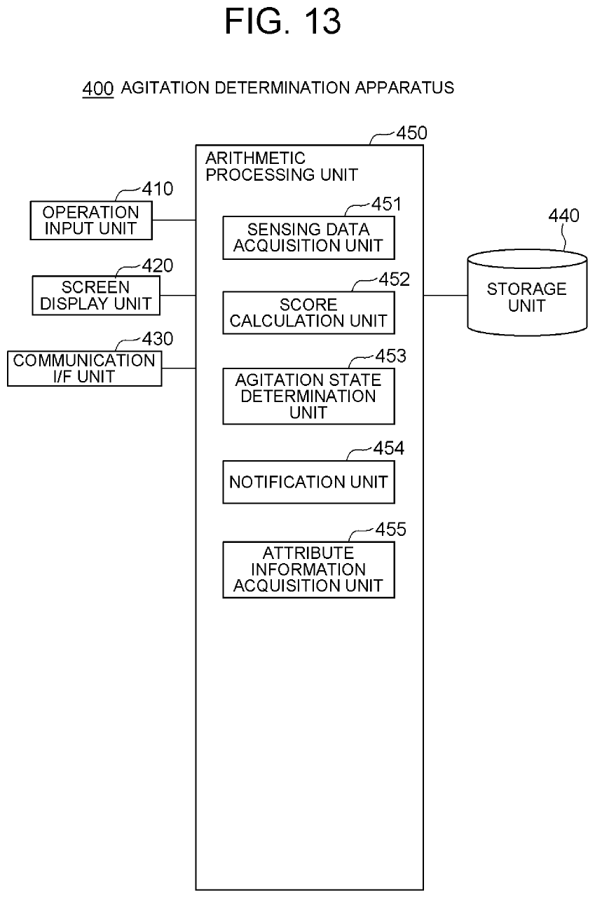

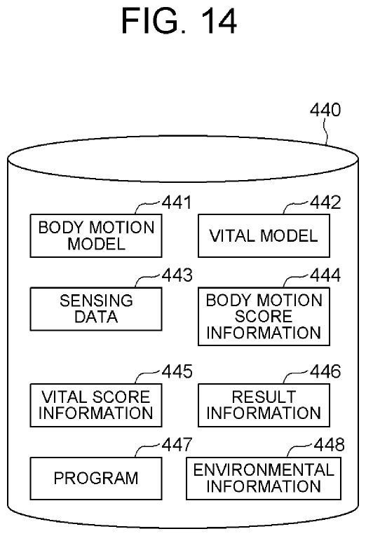

A first exemplary embodiment of the present disclosure will be described with reference to FIGS. 1 to 14. FIG. 1 is a diagram illustrating an example of the overall configuration of an agitation determination system 100. FIG. 2 is a block diagram illustrating an example of the configuration of a sensor apparatus 200. FIG. 3 is a block diagram illustrating an example of the configuration of a bed terminal 300. FIG. 4 is a block diagram illustrating an example of the configuration of an agitation determination apparatus 400. FIG. 5 is a diagram illustrating one example of information stored in a storage unit 440. FIG. 6 is a diagram illustrating one example of body motion data included in sensing data 443. FIG. 7 is a diagram illustrating one example of vital data included in the sensing data 443. FIG. 8 is a diagram illustrating one example of body motion score information 444. FIG. 9 is a diagram illustrating one example of vital score information 445. FIGS. 10 and 11 are diagrams for illustrating an example of processing by an agitation state determination unit 453. FIG. 12 is a flowchart illustrating examples of operations of the agitation determination apparatus 400. FIG. 13 is a block diagram illustrating another example of the configuration of the agitation determination apparatus 400. FIG. 14 is a diagram illustrating one example of information stored in the storage unit 440.

The first exemplary embodiment of the present disclosure will be described regarding the agitation determination system 100, which determines an agitation state of a patient wearing the sensor apparatus 200 based on data measured using the sensor apparatus 200. As will be described below, in the case of the present exemplary embodiment, the sensor apparatus 200 includes a body motion sensor 210, which measures body motion data such as an acceleration at a predetermined portion of the patient, and a vital sensor 220, which measures vital data such as a heart rate. Then, the agitation determination system 100 determines the agitation state of the patient based on a body motion score calculated based on the body motion data acquired by the body motion sensor 210, and a vital score calculated based on the vital data acquired by the vital sensor 220. By making the determination based on the body motion score and the vital score, the agitation determination system 100 can determine a predictive sign of agitation and also determine that the patient is agitated without overlooking it even when the patient is agitated without exhibiting the predictive sign of agitation. In other words, according to the above-described configuration, the agitation determination system 100 can accurately determine whether the patient is in the agitated state.

Note that the agitation determination system 100, which will be described in the present exemplary embodiment, can be utilized in various scenes, such as an acute hospital, a convalescent hospital, a care facility, and monitoring at home. In the following description, the present exemplary embodiment will be described citing an example when the agitation determination system 100 is utilized in a hospital such as an acute hospital or a convalescent hospital. Note that the agitation determination system 100 may be utilized under a condition requiring the agitation determination that is different from the above-described examples.

In the present exemplary embodiment, the agitation refers to a state in which the patient is restless and upset. The agitation may be caused by delirium or the like. Further, the agitation state indicates a state regarding the agitation of the patient. The agitation state indicates, for example, whether the patient is agitated or whether the patient exhibits the predictive sign of agitation. Note that the agitation state may include another index regarding a likelihood of the patient's agitation. When the patient is agitated, the patient may conduct a problematic behavior, such as falling off the bed, removing intubation, shouting abnormally, or committing violence. Therefore, it is desirable to accurately determine the agitation state.

FIG. 1 illustrates an example of the configuration of the agitation determination system 100. Referring to FIG. 1, the agitation determination system 100 includes, for example, the sensor apparatus 200, the bed terminal 300, and the agitation determination apparatus 400. As illustrated in FIG. 1, the sensor apparatus 200 and the bed terminal 300 are mutually communicably connected using short-range wireless communication such as Bluetooth (registered trademark), wired communication, or the like. Further, the bed terminal 300 and the agitation determination apparatus 400 are mutually communicably connected using short-range wireless communication such as Wi-Fi (registered trademark), wired communication, or the like. The bed terminal 300 and the agitation determination apparatus 400 may be connected via a relay apparatus such as a wireless base station.

Note that the number of sensor apparatuses 200, the number of bed terminals 300, and the number of agitation determination apparatuses 400 included in the agitation determination system 100 are not limited to the numbers exemplarily illustrated in FIG. 1. For example, the agitation determination system 100 can include a plurality of sensor apparatuses 200, a plurality of bed terminals 300, and/or a plurality of agitation determination apparatuses 400.

The sensor apparatus 200 is an apparatus including a sensor worn on at least one portion of the patient. The sensor apparatus 200 measures body motion data, which is a physical amount regarding the motion of the patient, and also measures vital data, which is a physical amount regarding the vital of the patient. FIG. 2 illustrates an example of the configuration of the sensor apparatus 200. Referring to FIG. 2, the sensor apparatus 200 includes, for example, a body motion sensor 210, a vital sensor 220, and a transmission/reception unit 230. For example, the sensor apparatus 200 can realize each of the above-described processing units by hardware. The sensor apparatus 200 may realize each of the above-described processing units through execution of a program stored in a storage device by an arithmetic device such as a CPU.

The body motion sensor 210 acquires time-series body motion data by measuring the body motion data. For example, the body motion sensor 210 includes at least one of an acceleration sensor, a gyro sensor (an angular velocity sensor), an angular sensor, a microphone, or the like.

Now, the body motion data refers to the physical amount regarding the motion of the patient's body. The body motion data includes, for example, an acceleration, an angular velocity, and/or an angle at a predetermined portion such as the patient's arm, body, or foot, and/or a vocal volume. The agitation determination apparatus 400, which will be described below, uses at least one of the acceleration, the angular velocity, or the angle at the predetermined portion of the patient, the vocal volume, or the like as the body motion data.

For example, in the case of the present exemplary embodiment, the body motion sensor 210 includes the acceleration sensor, and measures the acceleration at the predetermined portion of the patient. The body motion sensor 210 may measure body motion data different from the above-described examples as described above.

The vital sensor 220 acquires time-series vital data by measuring the vital data. For example, the vital sensor 220 includes at least one of a heart rate sensor, a respiratory rate sensor, a blood pressure sensor, a body temperature sensor, a blood oxygen saturation level sensor, or the like.

Now, the vital data refers to the physical amount varying according to the vital activity of the patient. The vital data includes, for example, a heart rate, a respiratory rate, a blood pressure value, a body temperature, a skin temperature, a blood flow rate, and/or a blood oxygen saturation level of the patient. The agitation determination apparatus 400, which will be described below, uses at least one of the heart rate, the respiratory rate, the blood pressure value, the body temperature, the skin temperature, the blood flow rate, the blood oxygen saturation level, or the like of the patient as the vital data.

For example, in the case of the present exemplary embodiment, the vital sensor 220 includes the heart rate sensor, and measures the heart rate of the patient. The vital sensor 220 may measure vital data different from the above-described examples as described above.

The transmission/reception unit 230 includes an antenna or the like, and transmits and receives data between the sensor apparatus 200 and the bed terminal 300. For example, the transmission/reception unit 230 transmits the body motion data that the body motion sensor 210 acquires by measuring it and the vital data that the vital sensor 220 acquires by measuring it to the bed terminal 300. Further, the transmission/reception unit 230 can transmit patient identification information for identifying the patient such as identification information provided to the sensor apparatus 200 in advance in association with the above-described body motion data and vital data.

This is an example of the configuration of the sensor apparatus 200. Note that the sensor apparatus 200 may be configured of one device or may be configured of a plurality of devices. For example, the sensor apparatus 200 can be configured of one device having functions as the body motion sensor 210 and the vital sensor 220. Alternatively, the sensor apparatus 200 may be configured of a plurality of devices such as a device having a function as the body motion sensor 210 and a device having a function as the vital sensor 220. The body motion sensor 210 may be configured of a plurality of devices. Further, the vital sensor 220 may be configured of a plurality of devices. Note that, in the case where the sensor apparatus 200 is configured of a plurality of devices, the plurality of devices may each include a transmission/reception unit.

The bed terminal 300 is an information processing apparatus set up in advance at, for example, a predetermined location such as around the bed on which the patient stays. The bed terminal 300, for example, transfers data. The bed terminal 300 is, for example, a smartphone, and has a screen display function. The bed terminal 300 may be an apparatus different from the smartphone. Note that the bed terminal 300 is a terminal set up at a predetermined location where the patient should stay or a predetermined location based on which a range where the patient should stay is defined, and is not limited to the terminal set up around the bed.

FIG. 3 illustrates an example of the configuration of the bed terminal 300. Referring to FIG. 3, the bed terminal 300 includes, for example, a transmission/reception unit 310 and a screen display unit 320. For example, the bed terminal 300 can realize each of the above-described processing units by hardware. The bed terminal 300 may realize each of the above-described processing units through execution of a program stored in a storage device by an arithmetic device such as a CPU.

The transmission/reception unit 310 includes an antenna or the like, and transmits and receives data between the bed terminal 300, and the sensor apparatus 200 and the agitation determination apparatus 400. The transmission/reception unit 310 receives, for example, the body motion data, the vital data, and the patient identification information transmitted from the sensor apparatus 200. Then, the transmission/reception unit 310 transmits, for example, the body motion data, the vital data, and the patient identification information received from the sensor apparatus 200 to the agitation determination apparatus 400. Further, the transmission/reception unit 310 can receive information indicating a result of the determination about the agitation state from the agitation determination apparatus 400.

The screen display unit 320 displays, for example, the body motion data, the vital data, the patient identification information, the information indicating the result of the determination about the agitation state received by the transmission/reception unit 310 on a screen. For example, the screen display unit 320 can display that the patient corresponding to the bed terminal 300 is in the agitated state on the screen based on, for example, the received information indicating the result of the determination about the agitation state.

The agitation determination apparatus 400 is an information processing apparatus that makes the determination based on the body motion data and the vital data measured by the sensor apparatus 200. The agitation determination apparatus 400 is set up at a predetermined location such as a nurse station. The agitation determination apparatus 400 is, for example, an information processing apparatus such as a personal computer, a tablet, or a smartphone used by medical staff such as a doctor or a nurse, an on-premise server set up in a hospital or the like, or a cloud server. The agitation determination apparatus 400 may be a combination of the information processing apparatus such as a personal computer, a tablet, or a smartphone, and the server and/or the like. For example, the agitation determination apparatus 400 is a combination of the on-premise server in the hospital and the smartphone, and makes the determination based on the body motion data and the vital data by the on-premise server and displays the result and/or notifies the medical staff of the result on the smartphone used by the medical staff. The agitation determination apparatus 400 may be an apparatus different from the above-described examples.

FIG. 4 illustrates an example of the configuration of the agitation determination apparatus 400. Referring to FIG. 4, the agitation determination apparatus 400 includes, for example, an operation input unit 410, a screen display unit 420, a communication I/F unit 430, a storage unit 440, and an arithmetic processing unit 450 as main constituent elements thereof.

The operation input unit 410 is configured of an operation input device such as a keyboard and/or a mouse. The operation input unit 410 detects an operation input by, for example, the nurse operating the agitation determination apparatus 400, and outputs it to the arithmetic processing unit 450.

The screen display unit 420 is configured of a screen display device such as an LCD (a Liquid Crystal Display). The screen display unit 420 can display various kinds of information stored in the storage unit 440, such as sensing data 443, body motion score information 444, vital score information 445, and result information 446, on the screen according to an instruction from the arithmetic processing unit 450. Note that the screen display unit 420 may be set up at a place remote from the place at which the arithmetic processing unit 450 and the like are set up. For example, only the screen display unit 420 among the configurations included in the agitation determination apparatus 400 may be set up at the nurse station. In this case, the arithmetic processing unit 450 and the like may be set up at a place different from the screen display unit 420, such as a server room.

The communication I/F unit 430 is configured of a data communication circuit. The communication I/F unit 430 performs data communication between the agitation determination apparatus 400 and an external apparatus such as the bed terminal 300 connected via wireless communication or the like or a mobile terminal carried by the nurse.

The storage unit 440 is a storage device such as a hard disk or a memory. FIG. 5 illustrates one example of information stored in the storage unit 440. As illustrated in FIG. 5, the storage unit 440 stores processing information required for various kinds of processing by the arithmetic processing unit 450, and a program 447 therein. The program 447 realizes the various kinds of processing units by being read in and executed by the arithmetic processing unit 450. The program 447 is read in from an external apparatus or a recording medium in advance via a data input/output function such as the communication I/F unit 430, and is stored in the storage unit 440. Examples of main information stored in the storage unit 440 include a body motion model 441, a vital model 442, the sensing data 443, the body motion score information 444, the vital score information 445, and the result information 446.

The body motion model 441 is a model for calculating a body motion score based on the body motion data measured by the sensor apparatus 200. For example, the body motion model 441 receives information according to the body motion data as an input and outputs the body motion score. The body motion model 441 is, for example, a trained model generated in advance by carrying out machine learning using a support vector machine (SVM), a neural network, or the like in an external apparatus or the like. For example, the machine learning is carried out by using data generated by labeling previously measured body motion data with the presence or absence of the agitation as supervisory data. The body motion model 441 is acquired from the external apparatus or the like via the communication I/F unit 430 or the like, and is stored in the storage unit 440.

In the case of the present exemplary embodiment, the body motion sensor 210 included in the sensor apparatus 200 measures the acceleration at the predetermined portion of the patient as described above. Therefore, the body motion model 441 can also be said to include an acceleration model for calculating the body motion score based on the data of the acceleration. Note that the body motion model 441 may include a plurality of types of models according to the type of the measured body motion data. For example, in a case where the agitation determination apparatus 400 uses the acceleration and the vocal volume as the body motion data, the body motion model 441 can include the acceleration model for calculating the body motion score based on the data of the acceleration and a vocal volume model for calculating the body motion score based on the data of the vocal volume. Further, in the case where the agitation determination apparatus 400 uses a plurality of pieces of body motion data, the body motion model 441 may include a model corresponding to all of the plurality of pieces of body motion data or may include a model corresponding to a part of the plurality of pieces of body motion data. For example, in the case where the agitation determination apparatus 400 uses the acceleration and the vocal volume as the body motion data to determine the agitation, the body motion model 441 may be one model for calculating the body motion score based on the data of the acceleration and the data of the vocal volume.

Note that the body motion score is an index for determining whether the patient is agitated. The body motion score is, for example, a value from 0 to 1, inclusive. The body motion score indicates a higher likelihood that the patient is agitated as the value thereof is closer to 1, and indicates a higher likelihood that the patient is not agitated as the value thereof is closer to 0. The body motion score may be an index expressed using two values that are 1 indicating that the patient is agitated and 0 indicating that the patient is not agitated. The body motion score may be an index indicating the intensity of the agitation, such as an index set to 2 for strong agitation and 1 for slight agitation.

Further, the data input to the body motion model 441 may be the time-series data itself of the body motion data such as the acceleration and/or the vocal volume, or may be various kinds of feature amounts calculated by subjecting the time-series data to processing for converting the time-series data into the feature amount such as averaging processing or differential processing. Further, the body motion model 441 may be configured to input one type of body motion data or may be configured to input a plurality of types of body motion data.

The vital model 442 is a model for calculating a vital score based on the vital data measured by the sensor apparatus 200. For example, the vital model 442 receives information according to the vital data as an input and outputs the vital score. The vital model 442 is, for example, a trained model generated in advance by carrying out machine learning using a support vector machine (SVM), a neural network, or the like on an external apparatus or the like. For example, the machine learning is carried out by using data generated by labeling previously measured vital data with the presence or absence of the agitation as supervisory data. The vital model 442 is acquired from the external apparatus or the like via the communication I/F unit 430 or the like, and is stored in the storage unit 440.

In the case of the present exemplary embodiment, the vital sensor 220 included in the sensor apparatus 200 measures the heart rate of the patient as described above. Therefore, the vital model 442 can also be said to include a heart rate model for calculating the vital score based on the data of the heart rate. Note that the vital model 442 may include a plurality of types of models according to the type of the vital data measured by the vital sensor 220, similarly to the example in the case of the body motion model 441. For example, in a case where the agitation determination apparatus 400 uses the heart rate and the respiratory rate as the vital data, the vital model 442 can include the heart rate model for calculating the vital score based on the data of the heart rate and a respiratory rate model for calculating the vital score based on the data of the respiratory rate. Further, in the case where the agitation determination apparatus 400 uses a plurality of pieces of vital data, the vital model 442 may include a model corresponding to all of the plurality of pieces of vital data or may include a model corresponding to a part of the plurality of pieces of vital data. For example, in the case where the agitation determination apparatus 400 uses the heart rate and the respiratory rate as the vital data to determine the agitation, the vital model 442 may be one model for calculating the body motion score based on the data of the heart rate and the data of the respiratory rate.

Note that the vital score is an index for determining whether the patient is agitated, and can be used to determine the predictive sign of agitation. The vital score is, for example, a value from 0 to 1, inclusive. The vital score indicates a higher likelihood that the patient will be agitated (i.e., the patient exhibits the predictive sign of agitation) as the value thereof is closer to 1, and indicates a higher likelihood that the patient will not be agitated (i.e., the patient does not exhibit the predictive sign of agitation) as the value thereof is closer to 0. The vital score may be an index expressed using two values that are 1 indicating that the patient exhibits the predictive sign of agitation and 0 indicating that the patient does not exhibit the predictive sign of agitation. The body motion score may be an index indicating the intensity of the agitation, such as an index set to 2 for strong agitation and 1 for slight agitation.

Further, the data input to the vital model 442 may be the time-series data itself of the vital data such as the heart rate, or may be various kinds of feature amounts calculated by subjecting the time-series data to processing for converting the time-series data into the feature amount such as averaging processing or differential processing. Further, the vital model 442 may be configured to input one type of vital data or may be configured to input a plurality of types of vital data.

The sensing data 443 includes data measured by the sensor apparatus 200. For example, the sensing data 443 includes the patient identification information, the body motion data, and the vital data in association with one another. In the case of the present exemplary embodiment, the body motion data includes the time-series data of the acceleration as described above. For example, FIG. 6 illustrates one example of the time-series data of the acceleration. In the case of FIG. 6, an x axis represents time and a y axis represents the magnitude of the acceleration. Further, the vital data includes the time-series data of the heart rate. For example, FIG. 7 illustrates one example of the time-series data of the heart rate. In the case of FIG. 7, an x axis represents time and a y axis represents the heart rate.

The body motion score information 444 includes the body motion score, which is the index for determining whether the patient is agitated. For example, the body motion score information 444 includes the patient identification information and the body motion score in association with each other.

FIG. 8 illustrates one example of the body motion score calculated by the score calculation unit 452 based on the body motion data illustrated in FIG. 6. In the case of FIG. 8, an x axis represents time and a y axis represents the body motion score. As illustrated in FIG. 8, the body motion score is expressed by, for example, a value from 0 to 1, inclusive. The body motion score indicates a higher likelihood that the patient is agitated as the value thereof is closer to 1, and indicates a higher likelihood that the patient is not agitated as the value thereof is closer to 0.

The vital score information 445 includes the vital score, which is the index for determining the predictive sign of agitation. For example, the vital score information 445 includes the patient identification information and the vital score in association with each other.

FIG. 9 illustrates one example of the vital score calculated by the score calculation unit 452 based on the vital data illustrated in FIG. 7. In the case of FIG. 9, an x axis represents time and a y axis represents the vital score. As illustrated in FIG. 9, the vital score is expressed by, for example, a value from 0 to 1, inclusive. The vital score indicates a higher likelihood that the patient will be agitated as the value thereof is closer to 1, and indicates a higher likelihood that the patient will not be agitated as the value thereof is closer to 0.

The result information 446 includes information indicating a result that the agitation state determination unit 453 determines based on the body motion score information 444 and the vital score information 445. For example, the result information 446 includes the patient identification information and the information indicating the result of the determination in association with each other.

The arithmetic processing unit 450 includes a microprocessor such as an MPU and a peripheral circuit thereof. The arithmetic processing unit 450 reads in the program 447 from the storage unit 440 and executes it, thereby causing the above-described hardware and program 447 to cooperate with each other to realize various kinds of processing units. Examples of main processing units realized by the arithmetic processing unit 450 include the sensing data acquisition unit 451, the score calculation unit 452, the agitation state determination unit 453, and the notification unit 454.

The sensing data acquisition unit 451 acquires the body motion data, the vital data, the patient identification information, and the like transmitted from the bed terminal 300 via the communication I/F unit 430. Then, the sensing data acquisition unit 451 stores the acquired body motion data and vital data into the storage unit 440 in association with the patient identification information as the sensing data 443.

The score calculation unit 452 calculates the body motion score using the body motion model 441 and also calculates the vital score using the vital model 442.

For example, the score calculation unit 452 acquires the body motion data including the time-series data of the acceleration like the example illustrated in FIG. 6 by referring to the sensing data 443. Further, the score calculation unit 452 inputs the acquired data to the body motion model 441, and calculates the body motion score at each time like the example illustrated in FIG. 8. After that, the score calculation unit 452 stores information indicating the calculated body motion score into the storage unit 440 as the body motion score information 444.

Further, the score calculation unit 452 acquires the vital data including the time-series data of the heart rate like the example illustrated in FIG. 7 by referring to the sensing data 443. Further, the score calculation unit 452 inputs the acquired data to the vital model 442, and calculates the vital score at each time like the example illustrated in FIG. 9. After that, the score calculation unit 452 stores information indicating the calculated vital score into the storage unit 440 as the vital score information 445.

Note that the score calculation unit 452 may input the time-series data itself to the body motion model 441 or the vital model 442, or may input various kinds of feature amounts calculated by subjecting the time-series data to the processing for converting the time-series data into the feature amount such as averaging processing or differential processing to the body motion model 441 or the vital model 442.

The agitation state determination unit 453 determines the agitation state of the patient based on the body motion score included in the body motion score information 444 and the vital score included in the vital score information 445. For example, the agitation state determination unit 453 determines whether the patient is agitated or whether the patient exhibits the predictive sign of agitation as the agitation state of the patient. Then, the agitation state determination unit 453 stores the result of the determination into the storage unit 440 as the result information 446. For example, the agitation state determination unit 453 stores information indicating such a result that the patient is determined to be agitated or information indicating such a result that the patient is determined to exhibit the predictive sign of agitation into the storage unit 440 as the result information 446.

For example, the agitation state determination unit 453 includes a body motion threshold value to be compared with the body motion score and a vital threshold value to be compared with the vital score in advance. Then, the agitation state determination unit 453 makes the determination based on the body motion score, the vital score, the body motion threshold value, and the vital threshold value. For example, the agitation state determination unit 453 determines that the patient is agitated if the body motion score is equal to or higher than the body motion threshold value. The agitation state determination unit 453 makes the determination based on the body motion score in this manner, thereby being able to determine that the patient is agitated when the patient is actually agitated. Further, for example, the agitation state determination unit 453 determines that the patient is agitated or the patient exhibits the predictive sign of agitation if the vital score is equal to or higher than the vital threshold value. Further, for example, the agitation state determination unit 453 determines that the patient exhibits the predictive sign of agitation if the vital score is equal to or higher than the vital threshold value even when the body motion score is lower than the body motion threshold value. The agitation state determination unit 453 may determine that the patient is in a normal state if the body motion score is equal to or lower than the body motion threshold value and the vital score is equal to or lower than the vital threshold value. In this manner, the agitation state determination unit 453 determines whether the patient is agitated or whether the patient exhibits the predictive sign of agitation based on the body motion score and the vital score.

More specifically, for example, in the case of the body motion score illustrated in FIG. 8, the body motion score becomes equal to or higher than the body motion threshold value during a period from 22:30 to slightly before 1:00, slightly before 2:00, around 4:30, and around 6:00 as illustrated in FIG. 10. Accordingly, the agitation state determination unit 453 determines that the patient is agitated during the period from 22:30 to slightly before 1:00, slightly before 2:00, around 4:30, and around 6:00.

Further, the agitation state determination unit 453 can determine that the patient exhibits the predictive sign of agitation if the vital score is equal to or higher than the vital threshold value even when the body motion score is lower than the body motion threshold value, as described above. Note that, in the case of the vital score illustrated in FIG. 9, the vital score becomes equal to or higher than the vital threshold value during the period from 22:30 to slightly before 1:00 and slightly before 2:00 as illustrated in FIG. 11. Therefore, even if the above-described period does not overlap the period during which the body motion score becomes equal to or higher than the body motion threshold value, the agitation state determination unit 453 can determine that the patient exhibits the predictive sign of agitation during the above-described period. Note that the agitation state determination unit 453 may be configured to also make the determination based on the vital score every time regardless of the result of the determination based on the body motion score.

Note that the values of the body motion threshold value and the vital threshold value may be set in any manner. For example, in the case of FIGS. 10 and 11, the values of the body motion threshold value and the vital threshold value are set to the same value. However, the values of the body motion threshold value and the vital threshold value may be values different from each other. Further, the body motion threshold value and the vital threshold value may be determined as appropriate according to, for example, attribute information of the patient, which will be described below.

Further, the agitation state determination unit 453 may consistently determine that the patient is agitated if the body motion score is equal to or higher than the body motion threshold value or if the vital score is equal to or higher than the vital threshold value. According to such a configuration, the medical staff can be notified of an abnormality in the patient without missing any of it.

Further, if being unable to use any one of the body motion score and the vital score to determine the agitation state of the patient, the agitation state determination unit 453 may determine the agitation state of the patient using the other of the scores alone. Now, being unable to use the body motion score to determine the agitation state of the patient includes, for example, being unable to acquire the body motion data by the sensor apparatus 200, and being unable to acquire the body motion data of the patient by the sensing data acquisition unit 451 due to a communication trouble or the like. Further, being unable to use the vital score to determine the agitation state of the patient includes, for example, being unable to acquire the vital data by the sensor apparatus 200, and being unable to acquire the vital data of the patient by the sensing data acquisition unit 451 due to a communication trouble or the like.

The notification unit 454 outputs the result of the determination made by the agitation state determination unit 453, for example, when the agitation state determination unit 453 determines that the patient is agitated. For example, when the agitation state determination unit 453 determines that the patient is agitated or exhibits the predictive sign of agitation, the notification unit 454 can display that the patient is agitated or that the patient exhibits the predictive sign of agitation on the screen display unit 420 together with the patient identification information of the determined patient. Further or alternatively, the notification unit 454 can transmit the determination that the patient is agitated or the like and the patient identification information of the patient to an external apparatus such as the bed terminal 300 relating to this patient or the mobile terminal carried by the nurse in charge of this patient. Note that the notification unit 454 may issue a notification in a manner different from the above-described examples, such as lighting a lamp at an entrance of a room in which the patient is hospitalized.

Note that the notification unit 454 may change the display content and/or the notification content according to a difference in the determination, i.e., whether the patient is determined to be agitated or determined to exhibit the predictive sign of agitation. For example, the notification unit 454 may use a method such as lighting a lamp in a different color according to the result of the determination when lighting the lamp at the entrance of the room in which the patient is hospitalized.

This is an example of the configuration of the agitation determination system 100. Note that the agitation determination apparatus 400 may be configured to determine whether to relearn the vital score based on the vital score, the body motion score, and/or the like. For example, the agitation determination apparatus 400 can be configured to relearn the vital score based on the body motion score if the patient cannot be determined to be agitated using the vital score because the vital score is lower than the vital threshold value and the body motion score is equal to or higher than the body motion threshold value. Subsequently, examples of operations of the agitation determination apparatus 400 will be described with reference to FIG. 12. Note that the order of the operations of the agitation determination apparatus 400 illustrated in FIG. 12 is one example, and the operations of the agitation determination apparatus 400 are not limited thereto.

The sensing data acquisition unit 451 acquires the body motion data, the vital data, the patient identification information, and the like transmitted from the bed terminal 300 via the communication I/F unit 430 (step S101).

The score calculation unit 452 calculates the body motion score using the body motion model 441 and also calculates the vital score using the vital model 442 (step S102). For example, the score calculation unit 452 calculates the body motion score by inputting the body motion data to the body motion model 441. Further, the score calculation unit 452 calculates the vital score by inputting the vital data to the vital model 442.

The agitation state determination unit 453 determines the agitation state of the patient based on the body motion score and the vital score. For example, if the body motion score is equal to or higher than the body motion threshold value (step S103), the agitation state determination unit 453 determines that the patient is agitated. On the other hand, if the body motion score is lower than the body motion threshold value (NO in step S103), the agitation state determination unit 453 confirms whether the vital score is equal to or higher than the vital threshold value. If the vital score is equal to or higher than the vital threshold value (YES in step S104), the agitation state determination unit 453 determines that the patient exhibits the predictive sign of agitation. On the other hand, if the vital score is lower than the vital threshold value (NO in step S104), the agitation state determination unit 453 determines that it is less likely that the patient is in the agitated state. Note that the processing in step S103 and the processing in step S104 may be performed in parallel.

If the agitation state determination unit 453 determines that it is highly likely that the patient will transition to the agitated state, the notification unit 454 issues a notification indicating the result of the determination made by the agitation state determination unit 453 (step S105). For example, the notification unit 454 can display that the patient is in the agitated state or that the patient exhibits the predictive sign of agitation on the screen display unit 420 together with the patient identification information of the patient determined to be in the agitated state. Further or alternatively, the notification unit 454 can transmit the determination that the patient is agitated or the like and the patient identification information of the patient to an external apparatus such as the bed terminal 300 relating to this patient or the mobile terminal carried by the nurse in charge of this patient.

These are examples of the operations of the agitation determination apparatus 400.

In this manner, the agitation determination apparatus 400 includes the score calculation unit 452, which calculates the body motion score and the vital score, and the agitation state determination unit 453. Due to such a configuration, the agitation state determination unit 453 can make the determination based on the body motion score and also make the determination based on the vital score. As a result, when the patient is actually agitated, the agitation determination apparatus 400 can determine that the patient is agitated without missing even the agitation accompanied by a low vital score. Accordingly, the agitation determination apparatus 400 can make the determination without overlooking the agitated state.

Note that, in the case where the body motion model 441 includes a plurality of models, a plurality of body motion scores may be calculated. In the case where a plurality of body motion scores is calculated in this manner, the agitation state determination unit 453 can determine that the patient is in the agitated state, for example, if even any one of the plurality of body motion scores becomes equal to or higher than the body motion threshold value. The agitation state determination unit 453 may make the determination by a method different from the above-described example, such as determining the agitation if more than half of the plurality of body motion scores become equal to or higher than the body motion threshold value.

Further, in the case where the vital model 442 includes a plurality of models, a plurality of vital scores may be calculated. In the case where a plurality of vital scores is calculated in this manner, the agitation state determination unit 453 can determine that the patient exhibits the predictive sign of agitation, for example, if even any one of the plurality of types of vital scores becomes equal to or higher than the vital threshold value. The agitation state determination unit 453 may determine that the patient exhibits the predictive sign of agitation if the plurality of vital scores satisfies a predetermined criterion, such as more than half of them being equal to or higher than the vital threshold value, all of them being equal to or higher than the vital threshold value, or an average value thereof being equal to or higher than the vital threshold value.

Further, the agitation state determination unit 453 may be configured to input the body motion data and the vital data to one model instead of inputting the body motion data to the body motion model 441 and the vital data to the vital model 442 or along with inputting the body motion data to the body motion model 441 and the vital data to the vital model 442, and determine the agitation state based on a score acquired as a result of the input. In other words, the agitation determination system 100 may be configured to calculate the body motion/vital score based on the body motion data and the vital data, and determine the agitation state using the calculated body motion/vital score. In this case, each of the body motion data and the vital data may be configured of only one type of data or may be configured of a plurality of types of data. In the case where the agitation determination system 100 is configured in this manner, the agitation determination apparatus 400 may include only one model (a body motion/vital model).

Further, the configuration of the agitation determination apparatus 400 is not limited to the example described with reference to FIG. 4. For example, FIG. 13 illustrates another example of the configuration of the agitation determination apparatus 400. Further, FIG. 14 illustrates another example of the information stored in the storage unit 440. Referring to FIG. 13, the arithmetic processing unit 450 of the agitation determination apparatus 400 can include, for example, attribute information acquisition unit 455.

The attribute information acquisition unit 455 acquires attribute information of the patient. For example, the attribute information acquisition unit 455 acquires medical record information of the patient from an external apparatus or the like, and acquires attribute information such as an age, a gender, and a paralysis state.

The attribute information acquired by the attribute information acquisition unit 455 can be utilized, for example, when a priority of the model or the score is determined. More specifically, the attribute information can be used to determine a priority serving as an index for determining which model or which score should be prioritized to make the determination, for example, in the case where the body motion model 441 or the vital model 442 includes a plurality of models or in the case where the plurality of body motion scores or vital scores is calculated.

For example, suppose that the body motion model 441 includes the acceleration model and the vocal volume model, and the vital model 442 includes the heart rate model. Further, suppose that the patient is determined to have hemiplegia at his/her hand and/or foot based on the attribute information acquired by the attribute information acquisition unit 455. In this case, the patient is expected to less move. In light thereof, the agitation state determination unit 453 can lower the priority of the acceleration model and raise the priorities of the vocal volume model and the heart rate model. On the other hand, if the patient is determined to be a patient capable of moving by him/herself to some degree based on the attribute information acquired by the attribute information acquisition unit 455, the agitation state determination unit 453 can, for example, set the same priority to all of the acceleration model, the vocal volume model, and the heart rate model. Note that the agitation state determination unit 453 may adjust the priority based on other attribute information.

Note that the above-described priority is the index indicating which model or which score should be prioritized to make the determination, for example, in the case where the body motion model 441 or the vital model 442 includes a plurality of models or in the case where a plurality of body motion scores or vital scores is calculated. The agitation state determination unit 453 can, for example, adjust the criterion when making the determination based on the priority. For example, if the patient is determined to have hemiplegia at his/her hand and/or foot based on the attribute information, the agitation state determination unit 453 can lower the priority of the acceleration model and raise the priories of the vocal volume model and the heart rate model, as described above. In this case, the agitation state determination unit 453 can make an adjustment of the criterion, such as making the determination using only the score based on the vocal volume model and the score based on the heart rate model, or adjusting the body motion threshold value to be compared with the score based on the acceleration model to a value different from the body motion threshold value to be compared with the score based on the vocal volume model. On the other hand, if the patient is determined to be a patient capable of moving by him/herself to some degree, the agitation state determination unit 453 can, for example, set the same priority to all of the acceleration model, the vocal volume model, and the heart rate model, as described above. In this case, the agitation state determination unit 453 can make an adjustment of the criterion, such as making the determination using all of the score based on the acceleration model, the score based on the vocal volume model, and the score based on the heart rate model, or setting the same value to all of the body motion threshold value and the vital threshold value. Note that the agitation state determination unit 453 may adjust the criterion in a manner different from the above-described examples.

Further, referring to FIG. 14, environmental information 448 can be stored in the storage unit 440 of the agitation determination apparatus 400.

The environmental information 448 indicates information such as a season and/or a temperature. The environmental information 448 is, for example, acquired from an external apparatus via the communication I/F unit 430 or input in advance using the operation input unit 410.

The information included in the environmental information 448 can also be utilized, for example, when the priority is determined, similarly to the attribute information acquired by the attribute information acquisition unit 455. For example, suppose that the body motion model 441 includes the acceleration model and the vocal volume model, and the vital model 442 includes the heart rate model, similarly to the above-described example. In this case, if the season is determined to be winter (or the temperature is determined to be low) based on the environmental information 448, the agitation state determination unit 453 can, for example, raise the priories of the vocal volume model and the heart rate model and lower the priority of the acceleration model because, for example, the patient tends to be slow-moving or a heart disease easily occurs. Accordingly, the agitation state determination unit 453 can, for example, make a correction to the body motion threshold value and/or the vital threshold value, such as adjusting the body motion threshold value to be compared with the score based on the acceleration model to a value different from the body motion threshold value to be compared with the score based on the vocal volume model.

The agitation determination apparatus 400 may include the environmental information 448 and/or the attribute information acquisition unit 455 in this manner by way of example.

Note that FIGS. 4 and 13 illustrate the example in which the functions as the agitation determination apparatus 400 are realized by one information processing apparatus. However, the functions as the agitation determination apparatus 400 may be realized by, for example, a plurality of information processing apparatuses connected via a network. Further, each configuration included in the agitation determination apparatus 400 may be set up at a remote place as described above.

### Second Exemplary Embodiment

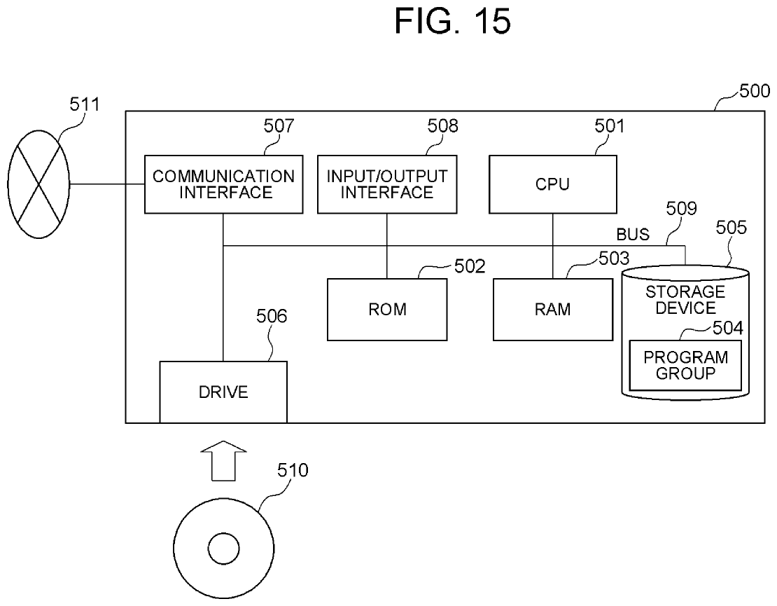

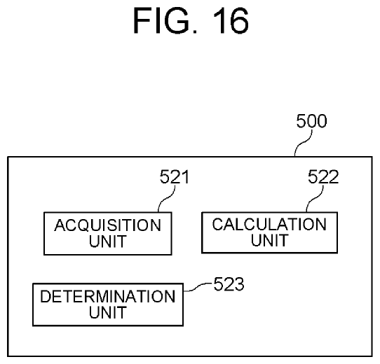

Next, a second exemplary embodiment of the present disclosure will be described with reference to FIGS. 15 and 16. The second exemplary embodiment will be described regarding an overview of the configuration of a determination apparatus 500.

FIG. 15 illustrates an example of the hardware configuration of the determination apparatus 500. Referring to FIG. 15, the determination apparatus 500 has the following hardware configuration as one example.
Central Processing Unit (CPU) 501 (arithmetic unit)
Read Only Memory (ROM) 502 (storage unit)
Random Access Memory (RAM) 503 (storage unit)
Program group 504 to be loaded to the RAM 503
Storage device 505 storing therein the program group 504
Drive 506 that performs reading and writing on a recording medium 510 outside the information processing apparatus
Communication interface 507 connecting to a communication network 511 outside the information processing apparatus
Input/output interface 508 for performing input/output of data
Bus 509 connecting the constituent elements

Further, the determination apparatus 500 can realize functions as an acquisition unit 521, a calculation unit 522, and a determination unit 523 illustrated in FIG. 16 through acquisition of the program group 504 by the CPU 501 and execution thereof by this CPU 501. Note that the program group 504 is, for example, stored in the storage device 505 or the ROM 502 in advance, and loaded to the RAM 503 or the like and executed by the CPU 501 as needed. Alternatively, the program group 504 may be provided to the CPU 501 via the communication network 511, or may be stored in the recording medium 510 in advance and read out by the drive 506 and provided to the CPU 501.

Note that FIG. 15 illustrates an example of the hardware configuration of the determination apparatus 500. The hardware configuration of the determination apparatus 500 is not limited to the above-described example. For example, the determination apparatus 500 may be configured of a part of the above-described configuration, such as not including the drive 106.

The acquisition unit 521 acquires body motion data according to a motion of a body of a target and vital data according to a state of the body of the target.

The calculation unit 522 calculates a body motion score serving as an index for determining an agitation state based on the body motion data acquired by the acquisition unit 521. Further, the calculation unit 522 calculates a vital score serving as an index for determining the agitation state based on the vital data acquired by the acquisition unit 521.

The determination unit 523 determines the agitation state of the target based on the body motion score and the vital score calculated by the calculation unit 522.

In this manner, the determination apparatus 500 includes the acquisition unit 521, the calculation unit 522, and the determination unit 523. Due to such a configuration, the determination unit 523 can make the determination based on the body motion score and also make the determination based on the vital score calculated by the calculation unit 522 based on the data acquired by the acquisition unit 521. As a result, when a patient is actually agitated, the determination apparatus 500 can determine that the patient is agitated without missing even the agitation accompanied by a low vital score. Accordingly, the determination apparatus 500 can make the determination without overlooking the agitated state.

Note that the above-described determination apparatus 500 can be realized by incorporating a predetermined program into a computer such as this determination apparatus 500. More specifically, a program according to another exemplary embodiment of the present invention is a program for causing the determination apparatus 500 to realize the acquisition unit 521, which acquires the body motion data according to the motion of the body of the target and the vital data according to the state of the body of the target, the calculation unit 522, which calculates the body motion score serving as the index for determining the agitation state based on the body motion data acquired by the acquisition unit 521 and calculates the vital score serving as the index for determining the agitation state based on the vital data acquired by the acquisition unit 521, and the determination unit 523, which determines the agitation state of the target based on the body motion score and the vital score calculated by the calculation unit 522.

Further, a determination method performed by a computer such as the above-described determination apparatus 500 is a method for causing the determination apparatus 500 to acquire body motion data according to a motion of a body of a target and vital data according to a state of the body of the target, calculate a body motion score serving as an index for determining an agitation state based on the acquired body motion data and calculate a vital score serving as an index for determining the agitation state based on the acquired vital data, and determine the agitation state of the target based on the calculated body motion score and vital score.

Even the invention of the program (or a recording medium) or the determination method configured in the above-described manner can also bring about advantageous effects equivalent to the above-described determination apparatus 500, thereby achieving the above-described object of the present invention.

### <Supplementary Notes>

The whole or part of the exemplary embodiments disclosed above can be described as, but not limited to, the following supplementary notes. Hereinafter, outlines of the determination apparatus and the like according to the present invention will be described. However, the present invention is not limited to the configurations described below.

### (Supplementary Note 1)

A determination apparatus comprising:
an acquisition unit configured to acquire body motion data according to a motion of a body of a target and vital data according to a state of the body of the target;
a calculation unit configured to calculate a body motion score serving as an index for determining an agitation state based on the body motion data acquired by the acquisition unit and calculate a vital score serving as an index for determining the agitation state based on the vital data acquired by the acquisition unit; and
a determination unit configured to determine the agitation state of the target based on the body motion score and the vital score calculated by the calculation unit.

### (Supplementary Note 2)

The determination apparatus according to supplementary note 1, wherein
the determination unit determines whether the target is agitated using the body motion score.

### (Supplementary Note 3)

The determination apparatus according to supplementary note 1 or 2, wherein
the determination unit determines whether the patient is agitated or whether the patient exhibits a predictive sign of agitation using the vital score.

### (Supplementary Note 4)

The determination apparatus according to supplementary note 3, wherein
the determination unit determines whether the target exhibits the predictive sign of agitation based on the vital score in a case where the target is determined not to be agitated based on the body motion score.

### (Supplementary Note 5)

The determination apparatus according to any one of supplementary notes 1 to 4, further comprising:
an attribute information acquisition unit configured to acquire attribute information indicating an attribute of the target, wherein
the determination unit determines the agitation state of the target based on the attribute information acquired by the attribute information acquisition unit, the body motion score, and the vital score.

### (Supplementary Note 6)

The determination apparatus according to supplementary note 5, wherein
the determination unit determines a priority of the score to use when determining the agitation state based on the attribute information acquired by the attribute information acquisition unit.

### (Supplementary Note 7)

The determination apparatus according to supplementary note 5 or 6, wherein
the determination unit adjusts a body motion threshold value to be compared with the body motion score based on the attribute information acquired by the attribute information acquisition unit.

### (Supplementary Note 8)

The determination apparatus according to any one of supplementary notes 1 to 7, wherein
the determination unit determines the agitation state of the target based on environmental information indicating an environment, the body motion score, and the vital score.

### (Supplementary Note 9)

The determination apparatus according to any one of supplementary notes 1 to 8, wherein
the body motion data includes data according to a plurality of types of motions of the body.

### (Supplementary Note 10)

The determination apparatus according to supplementary note 9, wherein
the body motion data includes at least acceleration data at a predetermined portion of the target and a vocal volume of the target.

### (Supplementary Note 11)

The determination apparatus according to supplementary note 9 or 10, wherein
according to the plurality of types of data included in the body motion data, the calculation unit calculates a plurality of types of the body motion scores according to the types of data included in the body motion data, and
the determination unit determines that the target is agitated in a case where any one of the plurality of body motion scores calculated by the calculation unit satisfies a predetermined criterion.

### (Supplementary Note 12)

The determination apparatus according to any one of supplementary notes 1 to 11, wherein
the calculation unit calculates a body motion/vital score based on the body motion data and the vital data, and
the determination unit determines the agitation state of the target using the body motion/vital score calculated by the calculation unit.

### (Supplementary Note 13)

A determination method for causing a computer to:
acquire body motion data according to a motion of a body of a target and vital data according to a state of the body of the target;
calculate a body motion score serving as an index for determining an agitation state based on the acquired body motion data;
calculate a vital score serving as an index for determining the agitation state based on the acquired vital data; and
determine the agitation state of the target based on the calculated body motion score and vital score.

### (Supplementary Note 14)

A recording medium recording a program for causing a computer to realize processing including:
acquiring body motion data according to a motion of a body of a target and vital data according to a state of the body of the target;
calculating a body motion score serving as an index for determining an agitation state based on the acquired body motion data;
calculating a vital score serving as an index for determining the agitation state based on the acquired vital data; and
determining the agitation state of the target based on the calculated body motion score and vital score.

Note that the program disclosed in each of the above-described exemplary embodiments and the supplementary notes is, for example, stored in a storage device or recorded in a computer-readable recording medium. For example, the recording medium is a portable medium such as a flexible disk, an optical disk, a magneto-optical disk, or a semiconductor memory.

While the present invention has been described with reference to the exemplary embodiments described above, the present invention is not limited to the above-described exemplary embodiments. The form and details of the present invention can be changed within the scope of the present invention in various manners that can be understood by those skilled in the art.

### REFERENCE SIGNS LIST

100 agitation determination system
200 sensor apparatus
210 body motion sensor
220 vital sensor
230 transmission/reception unit
300 bed terminal
310 transmission/reception unit
320 screen display unit
400 agitation determination apparatus
410 operation input unit
420 screen display unit
430 communication I/F unit
440 storage unit
441 body motion model
442 vital model
443 sensing data
444 body motion score information
445 vital score information
446 result information
447 program
448 environmental information
450 arithmetic processing unit
451 sensing data acquisition unit
452 score calculation unit
453 agitation state determination unit
454 notification unit
455 attribute information acquisition unit
500 determination apparatus
501 CPU
502 ROM
503 RAM
504 program group
505 storage device
506 drive
507 communication interface
508 input/output interface
509 bus
510 recording medium
511 communication network
521 acquisition unit
522 calculation unit
523 determination unit

What is claimed is:

1. A determination apparatus comprising:
at least one memory configured to store processing instructions; and
at least one processor configured to execute processing instructions to:
acquire body motion data according to a motion of a body of a target and vital data according to a state of the body of the target;
calculate a body motion score serving as an index for determining an agitation state based on the acquired body motion data and calculate a vital score serving as an index for determining the agitation state based on the acquired vital data; and
determine the agitation state of the target based on the calculated body motion score and the calculated vital score.

2. The determination apparatus according to claim 1, wherein
at least one processor configured to execute processing instructions to
determine whether the target is agitated using the body motion score.

3. The determination apparatus according to claim 1, wherein
at least one processor configured to execute processing instructions to
determine whether the target is agitated or whether the target exhibits a predictive sign of agitation using the vital score.

4. The determination apparatus according to claim 3, wherein
at least one processor configured to execute processing instructions to
determine whether the target exhibits the predictive sign of agitation based on the vital score in a case where the target is determined not to be agitated based on the body motion score.

5. The determination apparatus according to claim 1, wherein
at least one processor configured to execute processing instructions to:
acquire attribute information indicating an attribute of the target, and
determine the agitation state of the target based on the acquired attribute information, the body motion score, and the vital score.

6. The determination apparatus according to claim 5, wherein
at least one processor configured to execute processing instructions to:
determine a priority of the score to use when determining the agitation state based on the acquired attribute information.

7. The determination apparatus according to claim 5, wherein
the at least one processor configured to execute processing instructions to:
adjust a body motion threshold value to be compared with the body motion score based on the acquired attribute information.

8. The determination apparatus according to claim 1, wherein
at least one processor configured to execute processing instructions to:
determine the agitation state of the target based on environmental information indicating an environment, the body motion score, and the vital score.

9. The determination apparatus according to claim 1, wherein
the body motion data includes data according to a plurality of types of motions of the body.

10. The determination apparatus according to claim 9, wherein
the body motion data includes at least acceleration data at a predetermined portion of the target and a vocal volume of the target.

11. The determination apparatus according to claim 9, wherein the at least one processor configured to execute processing instructions to:
according to the plurality of types of data included in the body motion data, calculate a plurality of types of the body motion scores according to the types of data included in the body motion data, and
determine that the target is agitated in a case where any one of the calculated plurality of body motion scores satisfies a predetermined criterion.

12. The determination apparatus according to claim 1, wherein the at least one processor configured to execute processing instructions to:
calculate a body motion/vital score based on the body motion data and the vital data, and
determine the agitation state of the target using the calculated body motion/vital score.

13. A determination method for causing a computer to:
acquire body motion data according to a motion of a body of a target and vital data according to a state of the body of the target;
calculate a body motion score serving as an index for determining an agitation state based on the acquired body motion data;
calculate a vital score serving as an index for determining the agitation state based on the acquired vital data; and
determine the agitation state of the target based on the calculated body motion score and vital score.

14. A non-transitory computer readable recording medium recording a program for causing a computer to realize processing including:
acquiring body motion data according to a motion of a body of a target and vital data according to a state of the body of the target;
calculating a body motion score serving as an index for determining an agitation state based on the acquired body motion data;
calculating a vital score serving as an index for determining the agitation state based on the acquired vital data; and
determining the agitation state of the target based on the calculated body motion score and vital score.
